(19) United States
(12) Patent Application Publication
(10) Pub. No.: US 2006/0199230 A1
(43) Pub. Date: Sep. 7, 2006

(54) Use of prostate specific antigen to predict drug response

(75) Inventor: Stephen D. Mikolajczyk, San Diego, CA (US)

Correspondence Address: HOGAN & HARTSON LLP, 500 S GRAND AVE, SUITE 1900, LOS ANGELES, CA, 90071, US

(73) Assignee: Hybritech Incorporated

(21) Appl. No.: 11/072,854

(22) Filed: Mar. 3, 2005

(51) Int. Cl. G01N 33/574

(52) U.S. Cl. 435/7.23

(57) ABSTRACT

A method of determining the likelihood for a subject to respond to a drug, comprising: measuring at least one PSA-related value in a subject suffering from a PSA-related disorder and having been administered a drug for treating the PSA-related disorder; comparing the PSA-related value with its respective control value; and determining the likelihood for the subject to respond to the drug, wherein the PSA-related value is selected from the group consisting of the amount of BPSA, the amount of proPSA, the ratio of BPSA to proPSA, the ratio of BPSA to total PSA, the ratio of BPSA to free PSA, and a combination thereof; and wherein the amount of BPSA, the ratio of BPSA to proPSA, the ratio of BPSA to total PSA or the ratio of BPSA to free PSA, if higher than its respective control value by a respective first predetermined value; the amount of proPSA, if lower than its respective control value by a second predetermined value; or a combination thereof; indicates that the subject is likely responsive to the drug. Also disclosed is a kit for using such a method to predict whether a subject would be a drug responder.

### FIELD OF THE INVENTION

The present invention relates generally to the use of prostate specific antigen (PSA) as an indicator for drug response. In particular, the present invention relates to methods of predicting whether a subject would respond to drug treatments by determining changes in concentrations of various forms of PSAs and their ratios in the subject.

### BACKGROUND OF THE INVENTION

Throughout this application, various references are referred to within parentheses or cited directly. Disclosures of these publications in their entireties are hereby incorporated by reference into this application to more fully describe the state of the art to which this invention pertains. Full bibliographic citation for these references may be found at the end of this application, preceding the claims.

PSA is a member of the human kallikrein family of serine proteases (extensively reviewed in (1)). It is a serine endopeptidase with chymotrypsin-like enzymatic activity. The mature form of PSA has isoleucine as the N-terminal and 237 amino acid residues with a molecular mass of 28,400 D (2, 3).

PSA exists in the serum as the free form of PSA (free PSA, fPSA) but the majority of the PSA is in a complex with alpha-1-antichymotrypsin (ACT). It is generally accepted that the free PSA in serum is enzymatically inactive. PSA is a serine protease which is capable of complex formation with serum protease inhibitors. Human serum contains high levels of ACT and alpha-2-macroglobulin, both of which have been shown to complex with PSA (4). From 70% to 95% of the PSA in serun which can be detected by immunoassay is in a complex with ACT. The remainder is non-complexed, free PSA (5, 6).

It has been demonstrated that the level of free or non-complexed PSA in serum can improve the discrimination of prostate cancer from benign prostatic hyperplasia (BPH; 5, 7). An elevated ratio of free PSA to total PSA (free plus complexed PSA) is more highly correlated with BPH. Recent studies have identified different forms of PSA associated with the transition zone (TZ) and the peripheral zone (PZ) of the prostate. These zones are well defined in the art (8). Briefly, the TZ is characterized by small, simple glands embedded in a compact stroma, whereas the PZ is characterized by small glands embedded in a loose stroma. The TZ tissue forms a distinctive boundary with the PZ. The PZ and TZ are the zones of primary interest, since cancer is localized primarily to the PZ, while BPH is the result of tissue enlargement of the TZ. With extensive BPH, the TZ grows to several times the volume of other prostate zones. The TZ tissue surrounds the proximal prostate urethra, which is the reason that restricted urinary flow is often a symptom of enlarged TZ resulting from BPH.

Examination of PSA forms in tissues showed that free PSA consisted of 3 major forms (9): 1) precursor forms of PSA (proPSA), comprised of truncated proPSA forms which are elevated in prostate tumors and which have been shown to be good serum markers for detecting early and aggressive prostate cancer (10-12); 2) BPSA, which is elevated in nodular hyperplastic transition zone tissues (13), and which is elevated in the serum of men with BPH (14); 3) a third form of free PSA known as inPSA which is calculated by subtracting BPSA and all proPSA forms from the free PSA (9, 15). INPSA is thought to be composed of intact, inactive PSA that does not contain internally cleaved peptide bonds like BPSA, and does not contain pro leader peptides like the proPSA forms, and yet is enzymatically inactive and therefore does not form covalent complexes with serum protease inhibitors. Once the total proPSA, the BPSA and the total free PSA have been determined by immunoassay, inPSA is the remainder of the free PSA that is calculated by subtracting the proPSA and BPSA from the free PSA. Thus, free PSA contains proPSA; the truncated isoforms of proPSA which are more cancer-associated forms of PSA (12, 16); BPSA, a BPH-associated form of PSA; and inPSA. Studies have shown that proPSA can be used as a serum marker for cancer, and that BPSA is elevated in the serum of biopsy negative men (14).

Men with clinical BPH have medical symptoms related to urine flow such as restricted urine flow, incomplete bladder voiding, and the need to frequently urinate (17). Symptoms can be treated with drugs called 5-alpha-reductase inhibitors (5ARIs) and alpha adrenergic receptor antagonists commonly called alpha blockers. Alpha blockers work quickly by blocking prostatic adrenal receptors. Alpha blockers provide symptom relief within hours but do not affect the physiology of BPH progression, and so BPH can continue to progress over months and years until alpha blockers are no longer effective. On the other hand, 5ARIs work by blocking the conversion of testosterone to its physiologically active form, dihydrotestosterone (DHT). 5ARIs typically take 6 to 12 months for symptom relief but the drugs act by changing the biochemical pathways of testosterone activation in the prostate, thereby altering the course of disease progression. Without DHT, the primary active androgen in the prostate, many physiological pathways are affected. The reduced levels of DHT caused by administration of 5ARIs causes the prostate tissues to shrink, thereby providing eventual symptom relief for some men.

However, because 5ARIs must be taken for up to 12 months before beneficial effects may be fully realized, there are problems with patient compliance, in addition to the cost of drugs for 12 months for those men not found to benefit from 5ARI therapy. It is therefore desirable to have a marker to identify the men most likely to respond, or a marker to indicate who has responded biochemically to 5ARI therapy and who would be expected to receive eventual symptom relief related to shrinkage of the prostate. In current practice, alpha blockers and 5ARIs can initially be given together for combined short and long term symptom relief.

Another effect of 5ARIs is a drop in the PSA level to about half of what it was before drug administration since PSA expression is androgen regulated. Total PSA has been used as a correlate with prostate volume and therefore as a marker to stage which men should be given 5ARI therapy (18, 19). Administration of 5ARIs has been shown to decrease serum concentrations of PSA to about half of the pre-treatment level. However, the ability of total PSA to predict which men will respond to drug treatment is modest, and improved markers are needed.

### SUMMARY OF THE INVENTION

The present invention is based on an unexpected discovery that changes in concentrations of various PSA forms and their ratios in a subject correlate to the likelihood of the subject to respond to treatment with a drug. Thus, such changes can be used to predict whether the subject would be a drug responder.

Accordingly, one aspect of the invention provides a method of determining the likelihood for a subject to respond to a drug. The method comprises measuring at least one PSA-related value in a subject suffering from a PSA-related disorder and having been administered a drug for treating the PSA-related disorder; comparing the PSA-related value with its respective control value; and determining the likelihood for the subject to respond to the drug. The PSA-related value is selected from the group consisting of the amount of BPSA, the amount of proPSA, the ratio of BPSA to proPSA, the ratio of BPSA to total PSA, the ratio of BPSA to free PSA, and a combination thereof. If the amount of BPSA, the ratio of BPSA to proPSA, the ratio of BPSA to total PSA or the ratio of BPSA to free PSA is higher than its respective control value by a respective first predetermined value; the amount of proPSA is lower than its respective control value by a second predetermined value; or a combination thereof; the subject is likely responsive to the drug.

For example, the control value may be the PSA-related value in the subject measured prior to drug administration, and the PSA-related disorder may be benign prostatic hyperplasia, prostate cancer, or prostatitis.

The subject is responsive to the drug if the prostate volume of the subject is reduced by 10% or more 12 months after drug administration, the International Prostate Symptom Score (IPSS) of the subject is reduced by 2 or more 12 months after drug administration, or the maximal urine flow rate of the subject is increased by 1 cc or more 12 months after drug administration.

Drugs used for treating a PSA-related disorder include, but are not limited to, androgen-blocking drugs, such as 5-alpha-reductase inhibitors (SARIs), bicalutamine and flutamide. Examples of SARIs include, e.g., dutasteride and finasteride.

For the purpose of the present invention, a PSA-related value may be measured in a physiological fluid or prostate tissue sample from a subject. A physiological fluid sample may be, e.g., a sample of blood, serum, seminal plasma, urine or plasma.

In one embodiment, the first or second predetermined value is 5% or more. In another embodiment, the first or second predetermined value corresponds to a respective first or second predetermined odds ratio. For example, the first or second predetermined odds ratio may be 2 or more.

The PSA-related value may be measured at various time points, e.g., between 1 week and 3 months, after drug administration. For instance, the PSA-related value may be measured 1 month after drug administration. In one embodiment, if the first predetermined value is 86% or more for the amount of BPSA, 450% or more for the ratio of BPSA to proPSA, 78% or more for the ratio of BPSA to total PSA, 52% or more for the ratio of BPSA to free PSA; the second predetermined value is 48% or more; or a combination thereof; the subject is likely responsive to the drug.

In another embodiment, if the first predetermined value is 25% or more for the amount of BPSA, 230% or more for the ratio of BPSA to proPSA, 59% or more for the ratio of BPSA to total PSA, 44% or more for the ratio of BPSA to free PSA; the second predetermined value is 41% or more; or a combination thereof; the subject is likely responsive to the drug.

In still another embodiment, the subject is likely responsive to the drug if the odds ratio is 2 or more, which corresponds to a first predetermined value of 250% or more for the ratio of BPSA to proPSA, or a first predetermined value of 20% or more for the amount of BPSA.

The proPSA may be selected from the group consisting of [−1]proPSA, [−2]proPSA, [−3]proPSA, [−4]proPSA, [−5]proPSA, [−6]proPSA, [−7]proPSA, and a combination thereof.

In another aspect, the invention provides a kit comprising a first agent for measuring the amount of BPSA in a subject, a second agent for measuring the amount of free PSA in the subject, and a third agent for measuring the amount of total PSA or proPSA in the subject.

The invention further provides a kit comprising a first agent for measuring the amount of BPSA in a subject, a second agent for measuring the amount of proPSA in the subject, a third agent for measuring the amount of total PSA in the subject, and a fourth agent for measuring the amount of free PSA in the subject.

The kits of the present invention may be used to determine the likelihood for a subject to respond to a drug according to the method described above. Examples of the agents include, but are not limited to, antibodies, e.g., monoclonal or polyclonal antibodies.

The present invention provides methods and kits for predicting whether a subject would respond to a drug for treating a PSA-related disorder, which allow early selection of subjects for continued treatment with the drug.

The above-mentioned and other features of this invention and the manner of obtaining them will become more apparent, and will be best understood, by reference to the following description, taken in conjunction with the accompanying drawings. These drawings depict only a typical embodiment of the invention and do not therefore limit its scope.

### DESCRIPTION OF THE FIGURES

FIG. 1 is a plot showing the percentage change from baseline in the serum concentrations of total PSA (PSA), free PSA (fPSA), proPSA and BPSA at 1, 3 and 12 months after drug administration for the placebo group and the group treated with dutasteride, a 5-alpha-reductase inhibitor.

FIG. 2 is a plot showing the percentage change from baseline in the serum concentrations of total PSA (PSA), free PSA (fPSA), proPSA and BPSA at 1 month after drug administration for the placebo group and the group treated with dutasteride, a 5-alpha-reductase inhibitor.

FIG. 3 shows the relationship between the Odds Ratio of a response to drug treatment and the increase in BPSA.

### DETAILED DESCRIPTION OF THE INVENTION

The present invention is based on an unexpected discovery that BPSA is increased in the blood at about 1 month of SARI treatment and that this increase is more pronounced in men who will eventually respond to the drug treatment. Another surprisingly discovery was that proPSA shows a significant drop earlier than expected at about 1 month of SARI treatment and that this decrease is more pronounced in men who will eventually respond to the drug treatment. Unexpectedly, the ratio of BPSA to proPSA gives additional discrimination over either BPSA or proPSA alone.

As shown in Examples I and II below, the ratio of BPSA to proPSA at about 1 month after drug administration increased 450% over the baseline ratio in those men who showed 10% or greater decrease in prostate volume at 12 months post drug administration. The discrimination difference in the 1 month ratio from the baseline ratio is significantly greater than the individual increase in BPSA (86%) or the decrease in proPSA (48%). The ratios of BPSA to PSA and BPSA to free PSA were increased by 78% and 52%, respectively. When using an IPSS reduction of 2 or greater at 12 months post drug treatment as an indication of drug response, the increase in BPSA to proPSA ratio was 230% over baseline. There was no significant increase in BPSA (25%) though a significant decrease in proPSA (41%). The increases in BPSA/total PSA and BPSA/free PSA ratios were 59% and 44%, respectively. Clearly, total PSA and free PSA levels remain relatively constant and their ratio does not change significantly upon early drug treatment at about 1 month, and the levels decrease at 3 months due to DHT inhibition by the drug. Thus, a significant increase in BPSA, the BPSA to proPSA ratio, the BPSA to total PSA ration and the BPSA to free PSA ratio, and a significant decrease in proPSA were unexpected, and any of the changes may be significantly associated with drug response.

Accordingly, in one aspect, the invention provides a method of determining the likelihood for a subject to respond to a drug. The method comprises the following steps: (1) measuring at least one PSA-related value in a subject suffering from a PSA-related disorder and having been administered a drug for treating the PSA-related disorder; (2) comparing the PSA-related value with its respective control value; and (3) determining the likelihood for the subject to respond to the drug. The PSA-related value may be, e.g., the amount of BPSA, the amount of proPSA, the ratio of BPSA to proPSA, the ratio of BPSA to total PSA, the ratio of BPSA to free PSA, or a combination thereof. The subject is likely responsive to the drug if the amount of BPSA, the ratio of BPSA to proPSA, the ratio of BPSA to total PSA, or the ratio of BPSA to free PSA is higher than its respective control value by a respective first predetermined value; if the amount of proPSA is lower than its respective control value by a second predetermined value; or if any combination of the changes in values occurs in the subject.

The term “BPSA,” as used herein, refers to a form of PSA that comprises at least one clip at Lys 182 of the amino acid sequence of a mature form of PSA. The amino acid sequence of the mature form of PSA is fully described in reference (20), the relevant content of which is incorporated herein by reference. A mature form of PSA has 237 amino acid residues with a molecular mass of 28,400 D (21). A BPSA of the present invention has the same amino acid sequence of a mature form of PSA, except that the polypeptide chain of BPSA has been hydrolyzed between residues 182 and 183. In accordance with the embodiments of the present invention, a BPSA may also include one or more additional clips at Ile1, Lys145 and Lys146 of the amino acid sequence of a mature PSA. In one embodiment of the present invention, a BPSA consists of two clips at Lys145 and Lys182.

The term “proPSA,” as used herein, refers to a precursor form of PSA. A full-length precursor form of PSA includes a propeptide (i.e., leader peptide) of 7 amino acids, APLILSR, identified as SEQ ID NO: 1, which precedes the mature PSA protein of 237 amino acids. The full-length amino acid sequence of a proPSA is known in the art and is fully described in reference (20), the relevant content of which is incorporated herein by reference. For the purpose of the present invention, the last amino acid “R” of the propeptide sequence is counted as [−1] amino acid. For example, [−7] proPSA is a proPSA with its terminus starting at −7 a.a. of the propeptide. It contains the full-length proPSA. [−5] proPSA indicates that the terminus of the proPSA starts at −5 a.a. of the propeptide, and it contains the last five amino acid sequence of the propeptide sequence. For the purpose of the present invention, proPSA includes both full-length and truncated forms of proPSA with its terminus started at any amino acid of the propeptide, i.e, the proPSA may be [−1]proPSA, [−2]proPSA, [−3]proPSA, [−4]proPSA, [−5]proPSA, [−6]proPSA, [−7]proPSA, or a combination thereof.

For the purpose of the present invention, the term “total PSA” refers to all forms of PSA including free PSA and PSA complex with ACT. The term “free PSA,” as used herein, refers to PSA that is not complexed with ACT.

A “PSA-related value” refers to any value mathematically related to the quantity of any member of the PSA family, including, e.g., total PSA, proPSA, free PSA, and BPSA. Examples of PSA-related values are, but not limited to, the amount of BPSA, the amount of proPSA, the ratio of BPSA to proPSA, the ratio of BPSA to total PSA, the ratio of BPSA to free PSA, and the like.

A “control value” corresponding to a PSA-related value may be obtained, e.g., by measuring the respective value in a subject not suffering from a PSA-related disorder, or a subject that has not been administered a drug for treating the PSA-related disorder.

A “PSA-related disorder” refers to a disorder associated with elevated PSA levels in a subject compared to control subjects. For example, the elevated PSA level may be above 0.2 ng/ml of total PSA in blood, serum, or plasma. It may also include elevated levels of total PSA or one or more of the other forms of PSA such as BPSA or proPSA in a physiological fluid such as blood, serum, plasma, seminal plasma or urine, as well as in prostate tissue. Such disorders include, but are not limited to, BPH, prostate cancer, and prostatitis.

A subject is “likely responsive” to a drug or has a “likelihood” to respond to a drug when the subject has a statistically higher chance of responding to the drug than a control subject, e.g., a subject who has not been treated with the drug. For example, a subject is likely responsive to a drug when the odds ratio is 2 or more. “Odds” are the ratio of the number of subjects with an event (i.e., responding to a drug) in a group (a responder group or a control group) to the number of subjects without the event in the same group. An “odds ratio,” as used herein, refers to the ratio of the odds for the responder group to the odds for the control group.

The term “treating” is defined as administration of a drug with the purpose to cure, alleviate, relieve, remedy, or ameliorate a disorder, symptoms of the disorder, a disease state secondary to the disorder, or predisposition toward the disorder.

The term “drug,” as used herein, refers to a chemical entity or biological product, or a combination of chemical entities or biological products, administered to a person to treat a PSA-related disorder. The chemical entity or biological product may be a low molecular weight compound, or a larger compound, for example, an oligomer of nucleic acids, amino acids, or carbohydrates, including, without limitation, proteins, oligonucleotides, ribozymes, DNAzymes, glycoproteins, lipoproteins, and modifications and combinations thereof.

Any drugs that can be used to treat PSA-related disorders are within the invention. In particular, Examples I and II below describe the use of a 5-alpha-reductase inhibitor, dutasteride, for treating BPH. Another example of 5ARI is finasteride. Finasteride and dutasteride have some different pharmacokinetic paratmeters and biochemical properties, but both have the same specific effect of inhibiting the production of DHT (22, 23). Since the inhibition of DHT and the concomitant effect on prostate tissues and PSA production are considered to be the primary mechanism driving the unexpected rise in BPSA, the use of all 5ARIs are included in the present invention. While 5ARIs are used for the therapeutic treatment of BPH, a similar unexpected rise in BPSA and an earlier than expected drop in proPSA would be expected with drugs and treatments used for prostate cancer which inhibit androgen production, and thus testosterone and DHT production. In particular, drugs such as bicalutamine and flutaamide are used for complete androgen blockage and act similarly to castration in eliminating all physiological sources of androgen stimulation of the prostate. These drugs are used in cases of advanced prostate cancer and usually result in the complete elimination of PSA production by the prostate. It would be expected that, in these cases, the rise in BPSA or drop in proPSA would be even more pronounced or more rapid than with the 5ARIs. Thus, the unexpected rise in BPSA and rapid drop in proPSA in the current invention is associated with any drug which blocks adrogens such as testosterone or DHT and which has the effect of significantly lowering or eliminating the expression of PSA.

Any improvement in a parameter or symptom typically associated with a PSA-related disorder after administration of a drug for treating such disorder may be used to indicate response to the drug. For example, the decrease of prostate volume is known to correlate with BPH symptom reduction. Reduction in the International Prostate Symptom Score (IPSS) is another indicator for effective BPH treatment. As shown in Examples I and II below, the unexpected rise in BPSA, the unexpected early drop in proPSA, and the increased ratio of BPSA to proPSA, BPSA to total PSA, and BPSA to free PSA at about one month after drug treatment are associated with the decreases in prostate volume and IPSS at 12 months after drug treatment. Therefore, prostate volume decrease and IPSS reduction are useful indicators for response to drug treatment of BPH. Further more, increase of the maximal urine flow rate (Qmax) can also be used as an indicator for response to drug treatment of BPH.

The time point after drug administration at which the PSA-related values are measured may be determined using the method illustrated in the examples below or any other method known in the art. In an exemplary embodiment, subjects suffering from a PSA-related disorder may be identified in the judgment of the subjects or health care professionals. Such judgment may be either subjective (e.g., opinion) or objective (e.g., measurable by a test or diagnostic method). Prior to (e.g., 1 week before) administration of a drug for treating the PSA-related disorder, samples (e.g., blood) may be collected from the subjects and the PSA-related values are measured for each sample. The drug or a placebo is then administered to the subjects. At time intervals (e.g., every week between 1 week and 3 months after the drug administration), samples are collected again from the subjects and the PSA-related values are measured. The subjects treated with the drug are subsequently divided into a responder group and a non-responder group according to the criteria described above (e.g., decrease of prostate volume by at least 10% or reduction of IPSS by at least 2 at 12 months post drug administration for subjects suffering from BPH). The PSA-related values prior to and post drug administration are subjected to statistical analysis. The time point at which the PSA-related values are measured for predicting the likelihood for a subject to respond to the drug may be determined by choosing, e.g., a time point at which the change in any PSA-related value by administration of the drug is most significant in the responder group compared to the non-responder group or the placebo group, or an earliest time point at which the change in any PSA-related value is significantly different for the responder group compared to the non-responder group or the placebo group. Changes in PSA-related values are significantly different for the responder group compared to the non-responder group or the placebo group when the p value is, e.g., 0.05 or less.

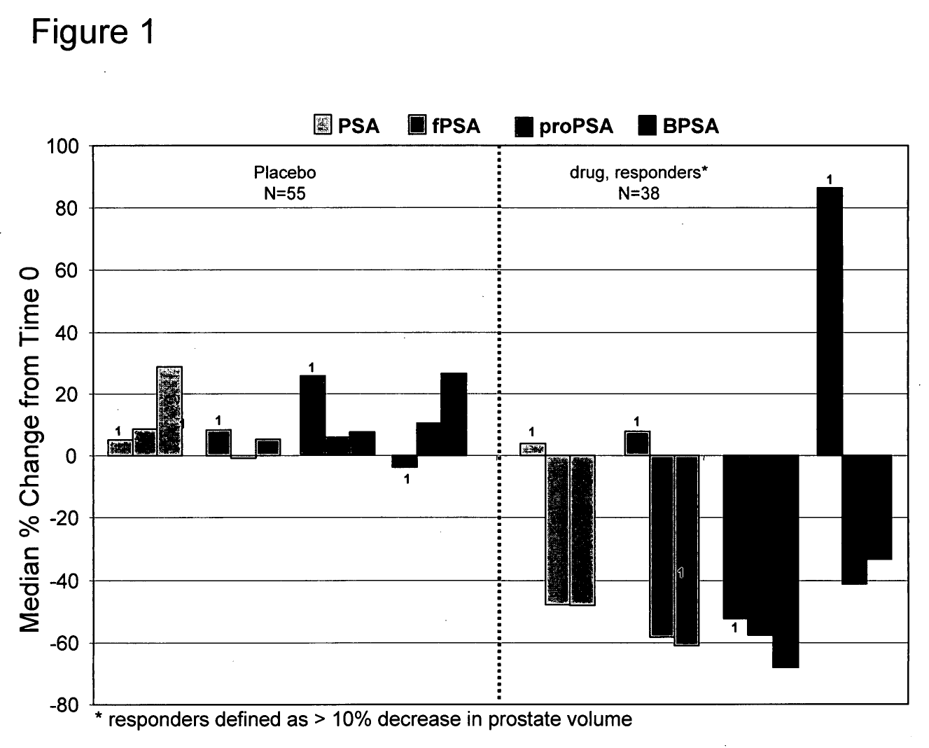

For example, the amount of BPSA, the amount of proPSA, or the ratio thereof, may be measured at about 1 month after administration of a 5ARI for predicting the likelihood for a subject to respond to the drug. As shown in Examples I and II below, a significant increase in the ratio of BPSA to proPSA about 1 month post drug administration indicates a response to the drug that is correlated to the decrease in prostate volume and BPH symptoms at 12 months post drug administration. The nominal time at about 1 month would ideally indicate 4 weeks of treatment but may include time periods up to 5 days before or after 4 weeks. However, in the broadest definition of the invention, meaningful determinations may be possible at any time from 1 week to three months post drug administration as indicated in other aspects of the invention described below. Since the inhibition of 5-alpha-reductase with 5ARIs is known to have some effect on levels of 5-alpha-reductase within 1 to 2 weeks after administration, it is reasonable to assume that there may be significant differences in the amplitude of the changes in the unexpected rise in BPSA serum level and the unexpectedly rapid decrease in proPSA concentration anywhere between 1 week and three months after the start of drug administration, the latter time where BPSA is shown in FIG. 1 to clearly decline below the baseline level. It would be anticipated that the rise in BPSA and fall in proPSA will begin at some time earlier than 1 month and will reach a maximum peak at some time between 1 week and 3 months, and that rates of rise and fall of the BPSA and proPSA respectively at different time points would provide different ratios and therefore different correlation parameters to the drug response observed at 12 months post drug administration. Therefore, the effective use of the unexpected rise in BPSA and fall in proPSA may encompass any early time period after which it is known that 5ARI has any effect on the production of DHT, where the BPSA is significantly higher than the baseline value before the start of drug treatment, but before the BPSA level falls below the baseline at 3 months.

Changes in PSA-related values are compared to their respective predetermined values (i.e., cutoff values) in order to predict the likelihood for a subject to respond to a drug. When the changes (increases or decreases) are equal to or above the predetermined values, the subject is predicted to be likely responsive to the drug. In general, the predetermined values may be at least 5% (e.g., 10%, 20%, 30%, 40%, 50%, 60%, 70%, 80%, 90%, 100%, 200%, 300%, 400%, 500%), with statistical significance.

The predetermined values may be obtained according to the method described below or any other methods known in the art. For example, changes in a PSA-related value can be measured in a responder group and a control group at a chosen time point after drug administration. A “control group” may be, e.g., a non-responder group or a group treated with a placebo. The changes are subjected to statistical analysis, e.g., Mann-Whitney test, to determine whether the changes in the responder group are significantly different from those in the control group. For example, when the p value is 0.05 or less, the average change in the PSA-related value in the responder group is statistically significantly different from that in the control group, and may be chosen as a predetermined value to help distinguish those who would responded to drug treatment.

For example, as shown in Example I below, an increase in the BPSA concentration of 86% at one month post drug administration is an indication of significant drug response for those who would realize a drop in prostate volume of 10% or greater at 12 months post drug administration (p=0.045, Table 1). Likewise, a decrease of 48% in proPSA and an increase of 450% in the ratio of BPSA to proPSA are statistically significant (p=0.0002 and <0.0001, respectively) and may therefore be used as cutoff values as an indication that these men have a likelihood of responding to drug treatment at 12 months.

The odds ratio of having a positive response to the drug may also be used to determine cutoff values as suitable indications for drug response. For example, a subject is likely responsive to a drug if the first or second predetermined value corresponds to a predetermined odds ratio of 2 or more (e.g., 3, 4, 5, 6, 7, 8, 9, 10).

For example, as shown in Example II below, the odds ratio of having an improvement of 2 in IPSS at 12 months post drug treatment is 2.27 if the BPSA to proPSA ratio is greater than 250% compared to ratio values less than 250%. Thus, in this case, the increase in BPSA to proPSA ratio by 250% at about one month may be viewed as a cutoff with significant clinical utility. The preferred embodiment of the present invention would use an odds ratio of at least 2 to establish a significant cutoff considered to have clinical utility.

Methods of measuring various forms of PSA are well known in the art. See, e.g., U.S. Pat. Nos. 6,423,503 and 6,482,599. For instance, the amount of BPSA, proPSA, total PSA or free PSA may be determined by any methods described herein or known in the art, or later developed, as long as they are capable of making such measurement. In accordance with one embodiment of the present invention, the amount of a particular PSA form (i.e., target PSA) contained in a sample may be measured by a method including the steps of contacting an agent that specifically binds to the target PSA with the sample under a condition that allows formation of a binary complex comprising the target PSA and the agent, and detecting and determining the amount of the complex as a measure of the amount of the target PSA.

For the purpose of the present invention, an agent may be any molecular species capable of binding to the target PSA with sufficient specificity. The binding specificity is sufficient if the binding allows the formation of a complex comprising the agent and the target PSA, and the determination of the amount of the complex. Examples of potential molecular species include, but are not limited to, antibodies, antigen-binding fragments derived from antibodies, and equivalents of antibodies, such as, but not limited to, aptamers, etc. For the purpose of the present invention, an agent specific for the target PSA may be selected by methods known in the art. For example, any known binding assays may be used to determine the specific binding activity of any given agent.

Immunohistochemical methods for detection of antigens in tissue specimens are well known in the art. For example, methods for the immunohistochemical detection of antigens are generally described in Taylor, Arch. Pathol. Lab. Med. 102:113 (1978). Briefly, in the context of the present invention, a tissue specimen obtained from a subject is contacted with an antibody, preferably a monoclonal antibody, recognizing a target PSA. In one embodiment of the present invention, the tissue specimen is a tissue specimen obtained from the prostate of a subject. The prostate tissue may be, e.g., a normal prostate tissue, a cancer prostate tissue, or a benign prostatic hyperplasia tissue.

Similarly, the general methods of in vitro detection of antigenic substances in fluid samples by immunoassay procedures are also well known in the art. For example, immunoassay procedures are generally described in Paterson et al., Int. J. Can. 37:659 (1986) and Burchell et al., Int. J. Can. 34:763 (1984). According to one embodiment of the present invention, an immunoassay for detecting a target PSA in a biological sample comprises the steps of: (a) contacting an amount of an agent which specifically binds to a target PSA with the sample under a condition that allows the formation of a binary complex comprising the agent and the target PSA and (b) determining the amount of the complex as a measure of the amount of the target PSA.

For the purpose of the present invention, the biological sample can be any physiological fluid sample that contains a target PSA. Examples of physiological fluid samples include, but are not limited to, blood, serum, seminal fluid, urine and plasma.

For the purpose of the present invention, both monoclonal antibodies and polyclonal antibodies may be used as long as such antibodies possess the requisite specificity for the antigen provided by the present invention. Preferably, monoclonal antibodies are used.

Monoclonal antibodies can be utilized in liquid phase or bound to a solid phase carrier. Monoclonal antibodies can be bound to many different carriers and used to determine a target PSA of the present invention. Examples of well-known carriers include glass, polystyrene, polypropylene, polyethylene, dextran, nylon, amylases, natural and modified celluloses, polyacrylamides, agaroses and magnetites. The nature of the carrier can be either soluble or insoluble for purposes of the invention. Examples of insoluble carriers include, but are not limited to, a bead and a microtiter plate. Those skilled in the art will know of other suitable carriers for binding monoclonal antibodies, or will be able to ascertain such under routine experimentation.

In addition, the monoclonal antibodies in these immunoassays can be detectably labeled in various ways. For example, monoclonal antibodies of the present invention can be coupled to low molecular weight haptens. These haptens can then be specifically detected by means of a second reaction. For example, it is common to use haptens such as biotin, which reacts with avidin, or dinitrophenyl, pyridoxal and fluorescein, which can react with specific antihapten antibodies. In addition, monoclonal antibodies of the present invention can also be coupled with a detectable label such as an enzyme, radioactive isotope, fluorescent compound or metal, chemiluminescent compound or bioluminescent compound. Furthermore, the binding of these labels to the desired molecule can be done using standard techniques common to those of ordinary skill in the art.

One of the ways in which the antibody can be detectably labeled is by linking it to an enzyme. This enzyme, in turn, when later exposed to its substrate, will react with the substrate in such a manner as to produce a chemical moiety which can be detected by, for example, spectrophotometric or fluorometric means (ELISA system). Examples of enzymes that can be used as detectable labels are horseradish peroxidase, malate dehydrogenase, staphylococcal nuclease, delta-5-steroid isomerase, yeast alcohol dehydrogenase, alpha-glycerophosphate dehydrogenase, triose phosphate isomerase, alkaline phosphatase, asparaginase, glucose oxidase, beta-galactosidase, ribonuclease, urease, catalase, glucose-6-phosphate dehydrogenase, glucoamylase and acetylcholine esterase. For increased sensitivity in the ELISA system, the procedures described can be modified using biotinylated antibodies reacting with avidin-peroxidase conjugates.

The amount of antigen can also be determined by labeling the antibody with a radioactive isotope. The presence of the radioactive isotope would then be determined by such means as the use of a gamma counter or a scintillation counter. Isotopes which are particularly useful are ³H, ¹²⁵I, ¹²³I, ³²P, ³⁵S, ¹⁴C, ⁵¹Cr, ³⁶Cl, ⁵⁷Co, ⁵⁸Co, ⁵⁹Fe, ⁷⁵Se, ¹¹¹N, ^{99m}Tc, ⁶⁷Ga and ⁹⁰Y.

The determination of the antigen is also possible by labeling the antibody with a fluorescent compound. When the fluorescently labeled molecule is exposed to a light of a proper wave length, its presence can then be detected due to fluorescence of the dye. Among the most important fluorescent labeling compounds are fluorescein isothiocyanate, rhodamine, phycoerythrin, phycocyanin, allophycocyanin, o-phthaldehyde and fluorescamine. Fluorescence-emitting metal atoms such as Eu (europium), and other lanthanides, can also be used. These can be attached to the desired molecule by means of metal-chelating groups, such as DTPA or EDTA.

Another way in which the antibody can be detectably labeled is by coupling it to a chemiluminescent compound. The presence of the chemiluminescent-tagged immunoglobulin is then determined by detecting the presence of luminescence that arises during the course of a chemical reaction. Examples of particularly useful chemiluminescent labeling compounds are luminol, isoluminol, aromatic acridinium ester, imidazole, acridinium salt, and oxalate ester.

Likewise, a bioluminescent compound may also be used as a label. Bioluminescence is a special type of chemiluminescence which is found in biological systems and in which a catalytic protein increases the efficiency of the chemiluminescent reaction. The presence of a bioluminescent molecule would be determined by detecting the presence of luminescence. Important bioluminescent compounds for purposes of labeling are luciferin, luciferase, and aequorin.

The quantitative determination of a target PSA of the present invention in a sample may be accomplished by competitive or non-competitive immunoassay procedures in either a direct or indirect format. Examples of such immunoassays are the radioimmunoassay (RIA) and the sandwich (immunometric) assay. The detection of the antigens using the monoclonal antibodies of the present invention can be done utilizing immunoassays which are run in either the forward, reverse or simultaneous modes, including immunohistochemical assays on physiological samples. Those skilled in the art will know, or can readily discern, other immunoassay formats without undue experimentation.

The term “immunometric assay” or “sandwich immunoassay” includes a simultaneous sandwich, forward sandwich, and reverse sandwich immnunoassay. These terms are well understood by those skilled in the art. Those skilled in the art will also appreciate that antibodies according to the present invention will be useful in other variations and forms of assays which are presently known or which may be developed in the future. These are intended to be included within the scope of the present invention.

Another aspect of the present invention provides a kit for determining the likelihood for a subject to respond to a drug according to the method described above. In one embodiment, the kit comprises a first agent for measuring the amount of BPSA in a subject, a second agent for measuring the amount of proPSA in the subject, and a third agent is for measuring the amount of total PSA or free PSA in the subject. In another embodiment, the kit comprises a first agent for measuring the amount of BPSA in a subject, a second agent for measuring the amount of total PSA in the subject, and a third agent for measuring the amount of free PSA in the subject. In still another embodiment, the kit comprises a first agent for measuring the amount of BPSA in a subject, a second agent for measuring the amount of proPSA in the subject, a third agent for measuring the amount of total PSA in the subject, and a fourth agent for measuring the amount of free PSA in the subject. The kit may further contains an insert with instructions indicating that a subject is likely responsive to a drug for treating a PSA-related disorder if the amount of BPSA, the ratio of BPSA to proPSA, the ratio of BPSA to total PSA, or the ratio of BPSA to free PSA is increased by a respective first predetermined value relative to its respective control value by administration of the drug; the amount of proPSA is decreased by a second predetermined value relative to its respective control value by administration of the drug; or a combination thereof.

Examples of the agents include, but are not limited to, antibodies, e.g., monoclonal or polyclonal antibodies. Antibodies to various forms of PSA are well known in the art. See, e.g., U.S. Pat. Nos. 6,423,503 and 6,482,599. The term “antibody” as used in this invention includes intact molecules as well as fragments thereof, such as Fab, F(ab′)₂ and Fv, which are capable of binding an epitopic determinant on a target PSA. These antibody fragments retain some ability to selectively bind with its antigen or receptor and are defined as follows: (1) Fab, the fragment which contains a monovalent antigen-binding fragment of an antibody molecule can be produced by digestion of the whole antibody with the enzyme papain to yield an intact light chain and a portion of one heavy chain; (2) Fab′, the fragment of an antibody molecule that can be obtained by treating the whole antibody with pepsin, followed by reduction, to yield an intact light chain and a portion of the heavy chain—two Fab′ fragments are obtained per antibody molecule; (3) F(ab′)₂, the fragment of the antibody that can be obtained by treating the whole antibody with the enzyme pepsin without subsequent reduction—F(ab′)₂ is a dimer of two Fab′ fragments held together by two disulfide bonds; (4) Fv, defined as a genetically engineered fragment containing the variable region of the light chain and the variable region of the heavy chain expressed as two chains; and (5) single chain antibody (“SCA”), defined as a genetically engineered molecule containing the variable region of the light chain, the variable region of the heavy chain, linked by a suitable polypeptide linker as a genetically fused single chain molecule. Methods of making these fragments are known in the art. (See, for example, Harlow and Lane, Antibodies: A Laboratory Manual, Cold Spring Harbor Laboratory, New York (1988), incorporated herein by reference.)

The following examples are intended to illustrate, but not to limit, the scope of the invention. While such examples are typical of those that might be used, other procedures known to those skilled in the art may alternatively be utilized. Indeed, those of ordinary skill in the art can readily envision and produce further embodiments, based on the teachings herein, without undue experimentation.

### EXAMPLES

### Materials and Methods

Patient Characteristics

A cohort of serum samples from men greater or equal to 50 years of age with BPH were used. The PSA range was from 1.5 to 10 ng/ml (median 3.2 ng/ml). International Prostate Symptom Score (IPSS) upon entry were 11-32 (moderate to severe, median 18.5). The median Qmax (urine flow rate in cc per second) was 9.3. Men received TRUS for prostate volume at time 0 and then received dutasteride in a blinded fashion at a rate of 0.5 mg per day. Serum was obtained at 0, 1, 3, and 12 months and was assayed for total PSA, proPSA, free PSA and BPSA. IPSS symptom scores were determined at 0, 1 and 12 months. Additional TRUS for prostate volume was performed at 12 months after the start of drug treatment.

### Example I

Association of Prostate Volume Decrease with Changes in Serum BPSA, proPSA, BPSA to proPSA Ratio, BPSA to Total PSA Ratio, and BPSA to Free PSA Ratio After Treatment with a 5-Alpha-Reductase Inhibitor, Dutasteride

Serum from placebo and drug-treated men were tested for levels of PSA forms at Time 0 (pretreatment baseline), and at 1, 3 and 12 months after the start of placebo or drug treatment. FIG. 1 shows the % change in total PSA (PSA), free PSA (fPSA), proPSA, and BPSA. In this plot, the decrease in prostate volume by 10% or more was used as the definition of drug response. Results show the placebo compared to the drug treatment: A) placebo-treated men whose prostate volume did not decrease by 10% or more after 12 months; B) drug-treated men whose prostate volume decreased by 10% or more after 12 months (drug-responders).

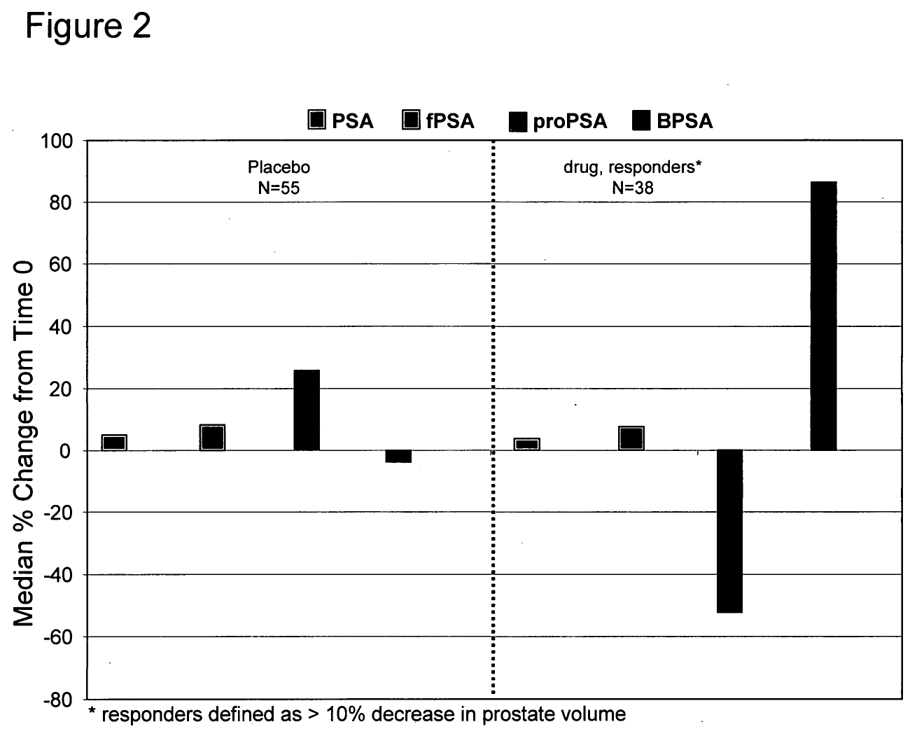

The major observation from this study was that the BPH-associated BPSA level was significantly increased at 1 month after drug treatment while the level of proPSA was significantly decreased. This is in contrast to total and free PSA levels which did not change significantly. FIG. 2 is a replot of FIG. 1 that only displays the 1 month changes in total PSA, free PSA, proPSA and BPSA for placebo and drug treatment. Table 1 shows a significant increase at 1 month (p<0.05) in the serum concentration (ng/ml) of BPSA, and as well as a significant decrease in proPSA (p=0.0002). The change in the ratio of BPSA to proPSA from baseline to 1 month showed the widest separation and highest significance (p<0.00001). There was no significant change in total PSA, free PSA, or the ratio of free to total PSA at 1 month corresponding to drug response defined as a decrease in prostate volume by 10% or more after 12 months of treatment (drug responders). There was no significant increase in BPSA in drug-treated men with less than 10% decrease in prostate volume at 12 months. There was no significant change in any PSA-related parameter with placebo.

Therefore, each of the parameters, the increase in BPSA, the decrease in proPSA, the increase in the ratio of BPSA to proPSA, the increase in the ratio of BPSA to total PSA, and the increase in the ratio of BPSA to free PSA, showed significant change at 1 month after the start of drug treatment significantly associated with the decrease in prostate volume after 12 months. No significant changes were observed in total PSA, free PSA and the ratio of free to total PSA.

TABLE 1
Significance (p-value, Mann-Whitney test) of the median change in
measurement between Time 0 and after 1 month of treatment with
drug or placebo in those men with a decrease of 10% or more
in prostate volume at 12 months.

| | +DRUG | | PLACEBO | |
|---|---|---|---|---|
| | Time 0, 1 | | Time 0, 1 | |
| Parameter | Month | p-value | Month | p-value |
| Total PSA | 3.02, 3.14 | 0.677 | 3.19, 2.73 | 0.863 |
| Free PSA | 0.70, 0.76 | 0.80 | 0.49, 0.44 | 0.73 |
| BPSA | 0.100, 0.186 | 0.0446 | 0.108, 0.079 | 0.962 |
| proPSA | 0.204, 0.097 | 0.0002 | 0.134, 0.151 | 0.423 |
| Free PSA/total PSA | 0.204, 0.214 | 0.56 | 0.175, 0.151 | 0.796 |
| BPSA/proPSA | 0.499, 2.26 | <0.0001 | 0.674, 0.470 | 0.423 |
| BPSA/total PSA | 0.028, 0.050 | 0.0014 | 0.0382, 0.0358 | 0.962 |
| BPSA/free PSA | 0.151, 0.230 | 0.0008 | 0.180, 0.201 | 0.605 |

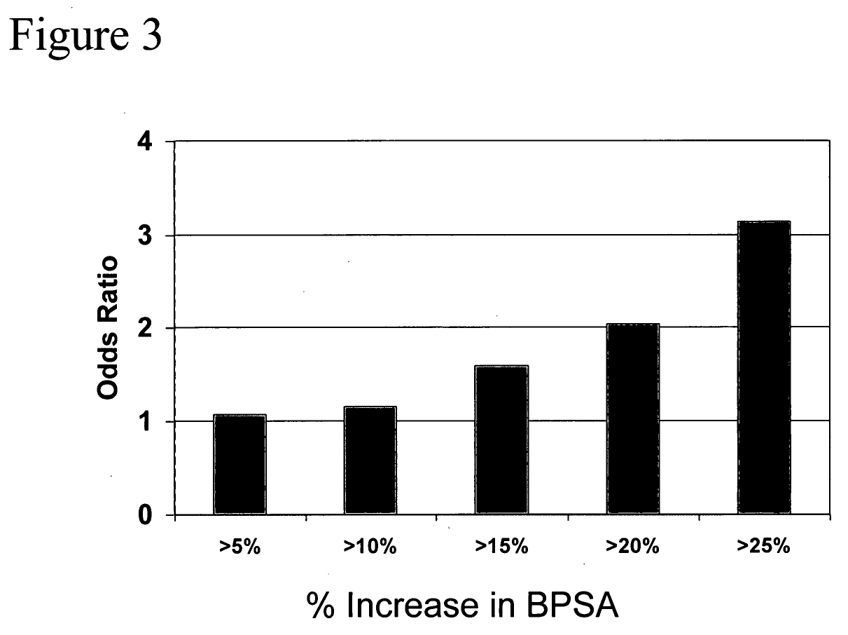

The Odds Ratio of a response to drug treatment is shown in FIG. 3. In this case, the odds of responding to the drug (drop of 10% or more in prostate volume at 12 months) increases with increasing serum BPSA concentration at 1 month. With a greater than 20% increase in BPSA concentration over the baseline at 1 month, there is a 2-fold increase in the odds of having a drug response at 12 months. A BPSA increase of greater than 25% shows a 3-fold likelihood of having a favorable drug response.

### Example II

Association of a Decrease in International Prostate Symptom Score (IPSS) with Changes in Serum BPSA, proPSA, BPSA to proPSA Ratio, BPSA to Total PSA Ratio, and BPSA to Free PSA Ratio After Treatment with a 5-Alpha-Reductase Inhibitor, Dutasteride

The concentrations of total PSA, free PSA, proPSA, and BPSA were examined at Time 0 (pretreatment baseline) and at 1 month after drug treatment. Drug response was defined as men who had improvement (decrease) in their IPSS of equal to 2 or greater after 12 months of treatment. Table 2 shows the increase in BPSA concentration at 1 month did not reach statistical significance in this sample set but the decrease in proPSA was significant (p=0.0002) as well as the increase in the ratio of BPSA to proPSA (p<0.0001). The increases in the ratio of BPSA to total PSA and the ratio of BPSA to free PSA were also highly significant. None of the placebo samples, or the men with IPSS improvement of less than 2 (drug non-responders), showed statistically a significant difference in any PSA-related parameter. However, the BPSA concentration after drug treatment was significantly elevated over the placebo at 1 month (Table 3). Similarly, the ratio of BPSA to total PSA and the ratio of BPSA to free PSA were also significantly increased over the placebo at 1 month of drug treatment. These results indicate that the increase in BPSA, decrease in proPSA, increase in the BPSA to proPSA ratio, increase in the BPSA to total PSA ration, and increase in the BPSA to free PSA ratio at 1 month after BPH drug treatment with 5ARI are significantly associated with those men who will respond to drug treatment at 12 months.

TABLE 2
Significance (p-value, Mann-Whitney test) of the median change in
measurement between Time 0 and after 1 month of treatment with
drug or placebo in those men with an improvement in International
Prostate Symptom Score of 2 or more at 12 months.

| | +DRUG | | PLACEBO | |
|---|---|---|---|---|
| | Time 0, 1 | | Time 0, 1 | |
| Parameter | Month | p-value | Month | p-value |
| Total PSA | 3.68, 3.14 | 0.261 | 2.89, 3.00 | 0.750 |
| Free PSA | 0.77, 0.67 | 0.55 | 0.675, 0.68 | 0.78 |
| BPSA | 0.120, 0.151 | 0.125 | 0.095, 0.091 | 0.476 |
| proPSA | 0.253, 0.103 | 0.0002 | 0.197, 0.236 | 0.667 |
| Free PSA/total PSA | 0.19, 0.21 | 0.399 | 0.205, 0.223 | 0.900 |
| BPSA/proPSA | 0.584, 1.32 | <0.0001 | 0.436, 0.444 | 0.978 |
| BPSA/total PSA | 0.029, 0.046 | 0.0027 | 0.030, 0.031 | 0.493 |
| BPSA/free PSA | 0.158, 0.228 | 0.0007 | 0.143, 0.157 | 0.250 |

TABLE 3
Significance (p-value, Mann-Whitney test) for the median value at 1
month of drug vs. placebo treatment in samples with a decrease of 2
or more in International Prostate Symptom Score. A p-value
<0.05 indicates that the measured BPSA, BPSA to total PSA ratio,
or BPSA to free PSA ratio is significantly different in the men
receiving drug compared to the placebo.

| Parameter | Placebo, Drug | p-value |
|---|---|---|
| Total PSA | 3.00, 3.08 | 0.80 |
| Free PSA | 0.62, 0.67 | 0.56 |
| BPSA | 0.091, 0.151 | 0.031 |
| Free PSA/total PSA | 0.22, 0.21 | 0.96 |
| BPSA/total PSA | 0.032, 0.046 | 0.013 |
| BPSA/free PSA | 0.158, 0.228 | 0.0017 |

Another method of determining a cutoff for clinical utility is to calculate the Odds Ratio of a response to drug treatment at 12 months, based on the percentage change in a measured parameter at 1 month. In this case, the odds of responding to the drug (drop of 2 or more in the IPSS at 12 months) increases with increasing ratio of BPSA to proPSA at 1 month. Calculations show that there is an Odds Ratio of 2.3 of having a response to drug at 12 months when the BPSA to proPSA ratio is >250% over baseline at 1 month compared to men whose BPSA to proPSA ratio is <250% increased at 1 month. With a >250% increase of BPSA to proPSA ratio over baseline at 1 month, there is at least a 2-fold increase in the odds of having a drug response at 12 months. An Odds Ratio of at least 2 is the preferred embodiment of the invention. Using Odds Ratio as a means to set a cutoff for the increase in the ratio of BPSA to proPSA would have a preferred cutoff of 250%.

A BPH symptom parameter that reflects some aspects of symptom score is the Qmax, or the maximum urine flow rate (cubic centimeters per second). The median value in this study population was 9.3 cc per second. Men who had a 25% increase in BPSA at 1 month after drug treatment had an Odds Ratio of 3.3 of having a 2 cc increase in flow rate at 12 months.

### Example III

Method for Determining the Diagnostic Utility of Changes in BPSA, proPSA, and the Ratio of BPSA to proPSA at Time Periods from 1 Week to 3 Months After Administration of a 5-Alpha-Reductase Inhibitor, Dutasteride

In the previous examples, the unexpected drop in proPSA and an increase in BPSA and the ratio of BPSA to proPSA at about 1 month were significantly associated with drug response at 12 months. However, 5ARIs begin to have effects on production of 5AR within 1-2 weeks of administration. Therefore, the unexpected rise in BPSA and the drop in proPSA associated with drug administration may have different rates of change and amplitudes at a time different from about 1 month. It is possible that there may be an optimum diagnostic utility for use of the relative rise in BPSA and the drop in proPSA to identify men who will respond to drug that is different from at about 1 month, as shown in Examles I and II. Using the data analysis as demonstrated in Examples I and II, one could reasonably design experiments in the same fashion, using the same concentration of drug and using the same or similar indicators of drug response in order to determine additional or improved diagnostic utility for the unexpected rise in BPSA and drop in proPSA. Since the biological mechanism for the rapid drop in proPSA is related to the inhibition of DHT production, this effect would be expected to affect all forms of proPSA. This is because the naturally expressed proPSA contains a 7-amino acid leader peptide and truncated proPSA forms, i.e., proPSA forms containing from 1 to 6 amino acids of the leader peptide, which are due to post translational protease activity. Therefore, the ratio of BPSA to proPSA may include any single form of native or truncated proPSA or the combined forms of proPSA, as shown in Examples I and II.

One experimental example of this method would include the following steps: 1) identify men meeting the patient entry criteria listed in the Materials and Methods section above; 2) take a sample of blood within 1 week prior to administration of dutasteride or finasteride; 3) take samples of blood at 1 week intervals from 1 week post drug administration to 3 months post drug administration; 4) measure BPSA and proPSA in these samples; 5) determine at which time points there is a statistically significant drop in proPSA, rise in BPSA, or increase in the BPSA to proPSA ratio compared to baseline values before administration of drug; 6) analyze the data as shown in Examples I and II as to the association of these parameters with drug response at from 6 to 12 months after drug administration; 7) drug response may be defined as a 10% or greater drop in prostate volume, a lowering of IPSS of 2 or more, an increase in urine flow (Qmax), or improvement in other quality of life parameters typically associated with symptomatic BPH; 8) determine the highest statistical correlation either by a) response mean or median of drop in proPSA, or increase in the ratio of BPSA to proPSA compared to placebo or controls or b) by Odds Ratio of having drug response, as shown in Examples I and II; 9) identify the optimum time after drug administration that can be used to identify the men who will respond to drug treatment at 12 months as calculated as shown in Examples I and II.

Additional considerations to apply towards identifying the optimum time after drug administration to be used to identify men who will respond to drug therapy are: a) the shortest time after drug administration when drug responders can be identified; b) maximum or highest predictive value as determined by Odds Ratio or other comparable statistical methods; c) times later than 1 month may be desirable if the drug administration is started at the same time as prostate biopsy for cancer identification. In the case of c), the prostate biopsy is known to cause a temporary rise in serum PSA levels due to the biopsy, and these PSA levels may interfere with BPSA testing. Therefore, in some cases, it may be desirable to perform BPSA and proPSA tests at time periods later than 1 month, but before 3 months, in order to calculate the ratio of BPSA to proPSA in order to choose an optimum time for men undergoing prostate biopsy.

The criteria for determining effective evaluation for diagnostic utility of the unexpected rise in BPSA, drop in proPSA, or increase in the ratio of BPSA to proPSA have been demonstrated in Examples I and II. These involve at least 2 different criteria: 1) The mean or median drop in proPSA, rise in the BPSA, or concomitant ratio with proPSA at a given time after drug administration for the population of men who responded to drug treatment at 12 months must be statistically significantly higher than that of the mean or median placebo control levels in those men who did not meet the drug response criteria. Using this criteria, one may establish a cutoff value for BPSA and proPSA at a given time period after drug administration that best helps to identify those men who will respond to drug treatment. 2) A second method as demonstrated in Examples I and II is to determine the percentage drop in proPSA, rise in BPSA, or concomitant ratio of BPSA to proPSA that gives at least a 2-fold Odds Ratio for a clinical drug response. In this case the cutoff is defined as the concentration or percent rise in BPSA or concomitant ratio of BPSA with proPSA that gives at least a 2-fold Odds Ratio. In Example II, the increase of 250% in the ratio of BPSA to proPSA gave an Odds Ratio of at least 2, the preferred embodiment of the current invention.

### REFERENCE LIST

1. Rittenhouse H G, Finlay J A. 1998. Mikolajczyk S D, Partin A W: Human kallikrein 2 (hK2) and prostate-specific antigen (PSA): Two closely related, but distinct, kallikreins in the prostate. Crit Rev Clin Lab Sci, 35:275-368.
2. Watt K W K, Lee P J, M'Timkulu T, Chan W P, Loor R. 1986. Human prostate-specific antigen: Structural and functional similarity with serine proteases. Proc Natl Acad Sci USA, 83:3166-3170.
3. Belanger A, van Halbeek H, Graves H C B, Grandbois K, Stamey T, Huang L H, Poppe I, Labrie F. 1995. Molecular mass and carbohydrate structure of porstate specific antigen: Studies for establishment of an international PSA standard. Prostate, 27:187-197.
4. Christensson A, Laurell C B, Lilja H. 1990. Enzymatic activity of prostate-specific antigen and its reactions with extracellular serine proteinase inhibitors. Eur J Biochem, 194:755-763.
5. Lilja H, Christensson A, Dahlen U, Matikainen M T, Nilsson O, Pettersson K, Lovgren T. 1991. Prostate-Specific Antigen in Serum Occurs Predominantly in Complex with α₁-antichymotrypsin. Clin Chem, 37(9):1618-1625.
6. Stenman U H, Leinonen J, Alfthan H, Rannikko S, Tuhkanen K, Alfthan O. 1991. A complex between prostate specific antigen and α₁-antichymotrypsin is the major form of prostate-specific antigen in serum of patients with prostatic cancer: assay of the complex improves clinical sensitivity for cancer. Cancer Res, 51:222-226.
7. Catalona W J. 1996. Clinical utility of measurements of free and total prostate-specific antigen (PSA): a review. Prostate, Supplement 7:64-69.
8. Bostwick D G, Dundore P A: Biopsy Pathology of the Prostate. Chapman & Hall, 1997.
9. Mikolajczyk S D, Marks L S, Partin A W, Rittenhouse H G. 2002. Free prostate-specific antigen in serum is becoming more complex. Urol, 59(6):797-802.
10. Mikolajczyk S D, Millar L S, Wang T J, Rittenhouse H G, Marks L S, Song W, Wheeler T M, Slawin K M. 2000. A precursor form of prostate-specific antigen is more highly elevated in prostate cancer compared with benign transition zone prostate tissue. Cancer Res, 60(3):756-759.
11. Catalona W J, Bartsch G, Rittenhouse H G, Evans C L, Linton H J, Amirkhan A, Horninger W, Klocker H, Mikolajczyk S D. 2003. Serum pro prostate specific antigen improves cancer detection compared to free and complexed prostate specific antigen in men with prostate specific antigen 2 to 4 ng/ml. J Urol, 170(6 Pt 1):2181-2185.
12. Catalona W J, Bartsch G, Rittenhouse H G, Evans C L, Linton H J, Horninger W, Klocker H, Mikolajczyk S D. 2004. Serum pro-prostate specific antigen preferentially detects aggressive prostate cancers in men with 2 to 4 ng/ml prostate specific antigen. J Urol, 171(6 Pt 1):2239-2244.
13. Mikolajczyk S D, Millar L S, Wang T J, Rittenhouse H G, Wolfert R L, Marks L S, Song W, Wheeler T M, Slawin K M. 2000. “BPSA,” a specific molecular form of free prostate-specific antigen, is found predominantly in the transition zone of patients with nodular benign prostatic hyperplasia. Urol, 55(1):41-45.
14. Linton H J, Marks L S, Millar L S, Knott C L, Rittenhouse H G, Mikolajczyk S D. 2003. Benign prostate-specific antigen (BPSA) in serum is increased in benign prostate disease. Clin Chem, 49(2):253-259.
15. Mikolajczyk S D, Rittenhouse H G. 2003. ProPSA and BPSA are forms of free prostate specific antigen in serum that help improve the detection of prostate cancer and benign disease (Japanese). Journal of Medical Technology, 47(9):981-988.
16. Mikolajczyk S D, Marker K M, Millar L S, Kumar A, Saedi M S, Payne J K, Evans C L, Gasior C L, Linton H J, Carpenter P, Rittenhouse H G. 2001. A truncated precursor form of prostate-specific antigen is a more specific serum marker of prostate cancer. Cancer Res, 61(18):6958-6963.
17. Kirby R S, Christmas T J: Benign Prostatic Hyperplasia. Second ed, Mosby International, 1997.
18. Roehrborn C G, Boyle P, Gould A L, Waldstreicher J. 1999. Serum prostate-specific antigen as a predictor of prostate volume in men with benign prostatic hyperplasia. Urol, 53(3):581-589.
19. Roehrborn C G, Boyle P, Bergner D, Gray T, Gittelman M, Shown T, Melman A, Bracken R B, deVere W R, Taylor A, Wang D, Waldstreicher J. 1999. Serum prostate-specific antigen and prostate volume predict long-term changes in symptoms and flow rate: results of a four-year, randomized trial comparing finasteride versus placebo. PLESS Study Group. Urol, 54(4):662-669.
20. Kumar A, Mikolajczyk S D, Groel A S, Millar L S, Saedi M S. 1997. Expression of Pro Form of Prostate-Specific Antigen by Mammalian Cells and Its Conversion to Mature, Active Form of Human Kallikrein 2. Cancer Res, 57:3111-3114.
21. Chen Z, Chen H, and Stamey T A. 1997. Prostate specific antigen in benign prostate hyperplasia: purification and characterization. J Urol, 157:2166-2170.
22. Andriole G, Bruchovsky N, Chung L W, Matsumoto A M, Rittmaster R, Roehrborn C, Russell D, Tindall D. 2004. Dihydrotestosterone and the prostate: the scientific rationale for 5-alpha-reductase inhibitors in the treatment of benign prostatic hyperplasia. J Urol, 172(4 Pt 1): 1399-1403.
23. Clark R V, Hermann D J, Cunningham G R, Wilson T H, Morrill B B, Hobbs S. 2004. Marked suppression of dihydrotestosterone in men with benign prostatic hyperplasia by dutasteride, a dual 5-alpha-reductase inhibitor. J Clin Endocrinol Metab, 89(5):2179-2184.

What is claimed is:

1. A method of determining the likelihood for a subject to respond to a drug, comprising:
measuring at least one PSA-related value in a subject suffering from a PSA-related disorder and having been administered a drug for treating the PSA-related disorder;
comparing the PSA-related value with its respective control value; and
determining the likelihood for the subject to respond to the drug,
wherein the PSA-related value is selected from the group consisting of the amount of BPSA, the amount of proPSA, the ratio of BPSA to proPSA, the ratio of BPSA to total PSA, the ratio of BPSA to free PSA, and a combination thereof; and
wherein the amount of BPSA, the ratio of BPSA to proPSA, the ratio of BPSA to total PSA or the ratio of BPSA to free PSA, if higher than its respective control value by a respective first predetermined value; the amount of proPSA, if lower than its respective control value by a second predetermined value; or a combination thereof; indicates that the subject is likely responsive to the drug.

2. The method of claim 1, wherein the control value is the PSA-related value in the subject measured prior to drug administration.

3. The method of claim 1, wherein the PSA-related disorder is benign prostatic hyperplasia.

4. The method of claim 3, wherein the subject is responsive to the drug if the prostate volume of the subject is reduced by 10% or more 12 months after drug administration.

5. The method of claim 3, wherein the subject is responsive to the drug if the International Prostate Symptom Score of the subject is reduced by 2 or more 12 months after drug administration.

6. The method of claim 3, wherein the subject is responsive to the drug if the maximal urine flow rate of the subject is increased by 1 cc or more 12 months after drug administration.

7. The method of claim 1, wherein the PSA-related disorder is prostate cancer or prostatitis.

8. The method of claim 1, wherein the drug is an androgen-blocking drug.

9. The method of claim 8, wherein the drug is selected from the group consisting of 5-alpha-reductase inhibitors (SARIs), bicalutamine and flutamide.

10. The method of claim 9, wherein the SARI is dutasteride or finasteride.

11. The method of claim 1, wherein the PSA-related value is measured in a physiological fluid or prostate tissue sample from the subject.

12. The method of claim 10, wherein the physiological fluid is selected from the group consisting of blood, serum, seminal plasma, urine and plasma.

13. The method of claim 1, wherein the first or second predetermined value is 5% or more.

14. The method of claim 1, wherein the first or second predetermined value corresponds to a respectively first or second predetermined odds ratio.

15. The method of claim 14, wherein the first or second predetermined odds ratio is 2 or more.

16. The method of claim 1, wherein the PSA-related value is measured between 1 week and 3 months after drug administration.

17. The method of claim 16, wherein the PSA-related value is measured 1 month after the drug administration.

18. The method of claim 17, wherein the first predetermined value is 86% or more for the amount of BPSA, 450% or more for the ratio of BPSA to proPSA, 78% or more for the ratio of BPSA to total PSA, 52% or more for the ratio of BPSA to free PSA, and the second predetermined value is 48% or more.

19. The method of claim 17, wherein the first predetermined value is 25% or more for the amount of BPSA, 230% or more for the ratio of BPSA to proPSA, 59% or more for the ratio of BPSA to total PSA, 44% or more for the ratio of BPSA to free PSA, and the second predetermined value is 41% or more.

20. The method of claim 17, wherein the first predetermined value is 250% or more for the ratio of BPSA to proPSA.

21. The method of claim 17, wherein the first predetermined value is 20% or more for the amount of BPSA.

22. The method of claim 1, wherein the proPSA is selected from the group consisting of [−1]proPSA, [−2]proPSA, [−3]proPSA, [−4]proPSA, [−5]proPSA, [−6]proPSA, [−7]proPSA, and a combination thereof.

23. A kit, comprising:
a first agent for measuring the amount of BPSA in a subject;
a second agent for measuring the amount of free PSA in the subject; and
a third agent for measuring the amount of total PSA or proPSA in the subject.

24. The kit of claim 23, wherein one or more of the agents are antibodies.

25. The kit of claim 24, wherein one or more of the antibodies are monoclonal or polyclonal antibodies.

26. A kit, comprising:
a first agent for measuring the amount of BPSA in a subject;
a second agent for measuring the amount of proPSA in the subject;
a third agent for measuring the amount of total PSA;
and a fourth agent for measuring the amount of free PSA in the subject.

27. The kit of claim 26, wherein one or more of the agents are antibodies.

28. The kit of claim 27, wherein one more of the antibodies are monoclonal or polyclonal antibodies.
